(19) United States
(12) Patent Application Publication
(10) Pub. No.: US 2026/0360537 A1
(43) Pub. Date: Nov. 12, 2026

(54) DETERMINING THE STATUS OF AN INDIVIDUAL IN RESPECT OF ALZHEIMER'S DISEASE

(71) Applicants: Neolys Diagnostics, Entzheim (FR); Alara Group, Entzheim (FR)

(72) Inventors: Sandrine Pereira, La Bastidonne (FR); Elise Berthel, Jons (FR); Fanny Carbillet, Ostwald (FR)

(21) Appl. No.: 19/142,315

(22) PCT Filed: Dec. 20, 2023
(86) PCT No.: PCT/EP2023/086928

§ 371 Date: Jun. 23, 2025

(30) Foreign Application Priority Data
Dec. 23, 2022 (FR) FR2214468

(51) Int. Cl. G16H 50/20; C12Q 1/6883; G16B 25/10

(52) U.S. Cl. CPC G16H 50/20; C12Q 1/6883; G16B 25/10; C12Q 2600/156; C12Q 2600/158

(57) ABSTRACT

The present invention relates to the field of diagnostics of neurodegenerative diseases. The present invention more particularly relates to a diagnostic method for evaluating the status (“predisposition before the subject develops the disease,” “evaluation of the disease,” and “predicted age of the onset of signs of the disease”) of a subject in respect of Alzheimer's disease, based on the analysis of the level of expression of qualified biomarkers.

### CROSS-REFERENCE TO RELATED APPLICATIONS

This application is a national phase entry under 35 U.S.C. § 371 of International Patent Application PCT/EP2023/086928, filed Dec. 20, 2023, designating the United States of America and published as International Patent Publication WO 2024/133443 A1 on Jun. 27, 2024, which claims the benefit under Article 8 of the Patent Cooperation Treaty of French Patent Application Serial No. FR2214468, filed Dec. 23, 2022.

### TECHNICAL FIELD

The present disclosure relates to the field of characterizing the predisposition and the risk of onset of neurodegenerative diseases. It more particularly relates to a method for evaluating the status based on indicators relating to predisposition before the subject develops the disease, early evaluation of the disease, or a probabilistic indicator of the predicted age of the onset of signs of the disease or of the age at which the patient will present a risk of developing the disease above a threshold value, and more generally a probabilistic indicator for a subject in respect of Alzheimer's disease, based on the analysis of the expression level of qualified biomarkers.

### BACKGROUND

The present disclosure relates to characterizing the predisposition of a subject to a neurodegenerative disease, and notably Alzheimer's disease. Degenerative diseases are diseases that develop progressively and that lead to physical, physiological or mental impairment in people affected.

These progressive and serious diseases include various diseases such as dementias (including Alzheimer's disease), Parkinson's disease, multiple sclerosis, paraplegia, myopathy or myasthenia gravis, epilepsy, stroke and other neurological conditions. The best-known degenerative diseases, and certainly the most widespread, are neurodegenerative diseases that cause degeneration of the nervous system.

These diseases result in a progressive loss of cognitive functions, with dementia affecting one in eight people under the age of 65. These diseases can affect people at an earlier age from 40 to 50 years old.

Degenerative diseases are generally genetic in origin, that is, they are inherited and transmitted from one generation to the next. But these diseases can also be caused by extensive and prolonged exposure to biological and toxic substances throughout life, or other disorders such as severe sleep apnea.

Early diagnosis of these diseases represents a major challenge in anticipating the onset of clinical signs and the onset of severe physical and mental disability, in order to help the subject organize a secure living environment adapted to the probable loss of autonomy, practice physical and intellectual exercises likely to delay the loss of autonomy, and slow the progression of the disease by organizing actions that can relieve or reduce some of the symptoms. Early diagnosis is the best way to stimulate the subject's cognitive and memory faculties, to anticipate and plan for the future while the subject still possesses all the faculties of discernment and decision-making.

However, to date, around 1 in 2 patients are unaware that they have the disease and will only learn of it at an advanced stage. Thus, nearly half a million people in France do not benefit from appropriate care. This has dramatic consequences for patients and their families.

For a long time, this diagnosis was made empirically, for example, by a general practitioner alerted by the patient's relatives to apparent disorders such as anosognosia, which is characterized by forgetfulness: the patient does not realize that he or she is in difficulty, and those around him or her encourage the patient to consult a doctor, a neurologist or a geriatrician in order to determine the origin of these disorders.

The usual diagnosis of a possible or probable degenerative disease is based substantially on clinical criteria and neuropsychological tests, which aim firstly to establish whether an individual presents a dementia syndrome, generally according to the criteria of the DSM IV (Diagnostic and Statistical Manual for Mental Disorders, published by the American Psychiatric Association), and then to determine the etiology of the dementia. The criteria for dementia essentially comprise the association of memory impairment and other cognitive functions impacting socio-professional activities and leading to a decline with respect to previous functioning. The diagnosis is strengthened by monitoring the patient's progress, which helps pinpoint the causes of dementia, notably by ruling out other causes of cognitive decline.

Efficient but highly invasive diagnostic methods have also been developed, based on the examination of brain tissue, which are mainly suited to post-mortem findings. Indeed, slices of brain tissue from Alzheimer's sufferers show the presence of amyloid in the form of extracellular protein nuclei, the neuritic plaques characteristic of Alzheimer's disease.

Research efforts have also focused on developing non-invasive in vivo methods using imaging techniques or biochemical or genomic methods to detect biomarkers.

Imaging techniques have been available for many years. Brain imaging, for example, can be used to assess regional hippocampal atrophy, which may also be present in other diseases affecting the elderly, and to exclude other causes of cognitive impairment, such as stroke sequelae.

For the imaging of brain diseases such as amyloid fibril or plaque-forming neurological diseases, a series of uncharged thioflavin-T derivatives have been developed as amyloid imaging agents and radiotracers that exhibit high affinity for amyloid deposits and high permeability across the blood-brain barrier. Extensive in vitro and in vivo studies of these amyloid imaging agents represented by thioflavin BTA-1 suggest that they bind specifically to amyloid deposits at concentrations typical of those detectable in positron emission tomography studies. In the complex environment of the human brain, non-specific binding of amyloid imaging compounds is low, even in control brains devoid of amyloid deposits.

The most eagerly awaited avenues involve a test that can be performed on a biological fluid taken from the subject, for example, a simple blood test.

A number of recent studies (O'Bryant, S. E., Xiao, G., Barber, R., Reisch, J., Hall, J., Cullum, C. M., Doody, R., Fairchild, T., Adams, P., Wilhelmsen, K., Diaz-Arrastia, R., & Texas Alzheimer's Research and Care Consortium (2011); A blood-based algorithm for the detection of Alzheimer's disease. Dementia and geriatric cognitive disorders, 32(1), 55-62. doi.org/10.1159/000330750) have proposed biomarker panels.

The test is performed on fasting blood samples collected in serum separator tubes, then allowed to coagulate at room temperature for 30 minutes, centrifuged, aliquoted and stored at −80° C. in plastic vials. Pooled samples from baseline or one-year follow-up examinations were thawed for analysis without additional freeze-thaw cycles using their human Multi-Analyte Profile (humanMAP) multiplexed immunoassay. Individual proteins were quantified using immunoassays on stained microspheres. Information on the least detectable dose, coefficient of variation between runs, dynamic range, overall recovery of the spiked standard and cross-reactivity with other analytes was obtained from other centers. However, these recent tests have proved non-reproducible.

Some commercial kits with specific biomarker panels are now available, the choice of which is based on studies outside Kings College London (Proteomics Sciences, Thambisetty et al., PLoS ONE, 6, 2001). However, these kits use expensive instruments such as mass spectrometry and, in some cases, require the use of CSF.

However, it is generally considered that only around 85% of cases of probable Alzheimer's disease are confirmed post-mortem. The 15% of false positives are generally due to other neurodegenerative diseases, such as frontotemporal dementia, notably including Lewy body dementia. Current methodologies for diagnosing neurodegenerative diseases therefore lack specificity.

More recently, certain biological markers have been discovered that appear to be specific to Alzheimer's disease.

Patent application US2012283114 proposes new sets of biomarkers for the diagnosis of Alzheimer's disease (AD) and related disorders comprising early diagnosis of MCI and early prodrome of AD, identification of disease subtypes, prediction of their response to disease management procedures, drugs and combinations thereof and monitoring of the response to these treatments, including validation of biomarkers for clinical trials. This method for detecting the presence or the risk of Alzheimer's disease in a mammal, or for facilitating the diagnosis and sub-classification of Alzheimer's disease, the method comprising determining the (relative) amount or presence, absence or alteration of a set of target biomarkers in a sample of biological fluid from the mammal and comparing the measured level of biomarker(s) to a reference value, where a deviation with respect to the reference value is indicative of the presence, risk, progression or severity of AD, and where the set of biomarkers comprises a protein or a ribonucleic acid (RNA) encoded by each of the following genes: APBA1, ATG7, BECN1, CD44, CDH2, COL18A1, ERBB4, F3, FLNA, FYN, GRIN2B, IL20, ITPR1, LRP8, MTOR, NPPC, NRP1, PDGFC, ROB01, SEMA3E, TGFB1, THBS1, VEGFR1 and WWOX.

Patent application EP2924124 relates to the use of mitochondrial DNA as a quantitative biomarker for neurodegenerative diseases, preferably Alzheimer's disease, as well as a method and a kit for diagnosing and/or prognosing these diseases using this biomarker.

Patent application US2021/311076 relates to methods, compositions and uses for counteracting the effects of aberrant lymphatic drainage of the meninges and/or for modulating the lymphatic vessels of the central nervous system. In certain embodiments, the present disclosure relates to methods, compositions and uses for treating, preventing or improving symptoms of a neurodegenerative disease associated with aberrant lymphatic drainage of the meninges. Modulation of lymphatic vessels, or neutralization of the effects of aberrant meningeal lymphatic drainage, according to some embodiments, are used to diagnose, treat, prevent or improve symptoms of neurodegenerative diseases such as Alzheimer's disease (AD) and dementia.

The article by Huabin Zhao et al.: “Identification ferroptosis-related hub genes and diagnostic model in Alzheimer's disease,” Front. Mol. Neuroscie 16:1280639 (2023), presents the identification of ferroptosis process genes involved in Alzheimer's disease from an in silico analysis of data available on the GEO database (Barette et al., 2013). This article describes the use of mathematical models to evaluate these biomarkers.

The article by the authors J. E. McDermott et al.: “Challenges in biomarker discovery: combining expert insights with statistical analysis of complex amies data,” EXPERT OPINION ON MEDICAL DIAGNOSTICS, vol. 7, no. 1, Jan. 18, 2013(2013 -01-18), pages 37-51, XP093095314, presents different statistical approaches to assembling biomarker panels focused on the use of sophisticated statistical models on large quantitative data sets. This article describes possible approaches to this problem, including metrics for biomarker assessment.

### BRIEF SUMMARY

In order to overcome these disadvantages, the present disclosure relates in its most general sense to a method for automatically classifying the status of a subject in respect of Alzheimer's disease.

This classification translates, for example, into a category representing the predisposition before the subject develops the disease, the evaluation of the disease, the predicted age of the onset of signs of the disease, and the age at which the probability of the onset of signs of the disease exceeds a threshold value.

The method comprises the following steps:
A. an initial step consisting of collecting reference data comprising:
   a) a database of reference control values VTₓ for healthy subjects for each of the ATM, APOE, GSNOR, PARKIN2, STOX2, mitochondrial DNA genes; and
   b) known status classes, determined from normalized n-tuples CRₓ obtained from biological samples from homogeneous cohorts with respect to one or more patient classification criteria having a common reference status R to determine a subspace R corresponding to a given status;
B. processing steps, for a new subject, consisting of:
   c. performing a quantification (by a known technique, for example, PCR or western blot) on genomic material of the new subject comprising at least some of the following markers:
      of the ATM gene and its expression to establish an initial value VATM;
      of the APOE gene and its expression to establish a second value VAPOE;
      of the GSNOR gene and its expression to establish a third value VGSNOR;
      of the PARKIN2 gene and its expression to establish a fourth value VPARKIN2;
      of the STOX2 gene and its expression to establish a fifth value VSTOX2; and
      of mitochondrial DNA to establish a sixth value VADNM;
   d. normalizing the values Vₓ to determine six normalized coefficients Cₓ=I Vₓ−VTₓI/VTₓ; and
   e. determining the nearest subspace (in the sense of Euclidean distance) of the n-tuple Cₓ to determine the status of the subject.

The result obtained is the automatic obtaining of the automatic classification of the status representative of a subject in respect of the predisposition to future Alzheimer's disease. The status is characterized by the subject belonging to a class, determined according to a classification model of different predisposition situations established by a community of users of the invention. These classes (“predisposition before the subject develops the disease,” “evaluation of the disease,” and “predicted age of the onset of signs of the disease”) of a subject in respect of Alzheimer's disease correspond to a subset of subjects having a similar predisposition status to Alzheimer's disease, the whole population being segmented into a number of classes decided by the community of users of the invention. This segmentation can be determined:
based on the probability of Alzheimer's disease occurring at any given time, with classes ranging from “zero probability” to “certainty”;
based on the length of time after which the probability of developing Alzheimer's disease exceeds a threshold;
or any other segmentation method.

By “control values VTₓ,” a measurable indicator is meant, notably:
the expression level of the gene of interest (typically zero, low, medium, high);
the expression level of the protein of interest;
the methylation profile of the gene of interest;
and more generally any indicator known to a person skilled in the art.

Biological samples are blood samples, or skin samples or brain cell samples or cells originating from a lumbar puncture.

The method according to the present disclosure optionally comprises an additional step consisting of isolating the cells to characterize the morphology of the cell nuclei and the progerin level, to determine the P value of the proportion of cells having a deformed nucleus and to determine the aging status based on the class to which this P value belongs, to be weighted by analyzing the morphology of the nuclei (80% of normal nuclei) by immunofluorescence and antibodies and/or a fluorescent DNA intercalating dye, to confirm the probability of AD, even asymptomatic, and/or a higher than normal progerin level.

According to a variant, the method comprises:
a cell analysis step to verify the perinuclear presence of phosphoAPOE protein and/or to quantify the phosphoAPOE protein.
and/or an analysis step using a regression method on an N-tuple of values Vx corresponding to the subject to be diagnosed and an N-tuple of control values VTx.
and/or a step of assaying a pATM-pAPOE biomarker in a skin sample from the subject.
and/or a step of quantifying the percentage of fibroblasts showing spontaneous and/or post-irradiation perinuclear accumulations of pATM and pAPOE on a significant sample of cells.

The present disclosure also relates to a kit for evaluating the status (“predisposition before the subject develops the disease,” “evaluation of the disease,” and “predicted age of the onset of signs of the disease”) of a subject in respect of Alzheimer's disease, in accordance with the method referred to in claim 1, wherein it comprises at least some of the following markers:
of the ATM gene to establish an initial value VATM;
of the APOE gene to establish a second value VAPOE;
of the GSNOR gene to establish a third value VGSNOR;
of the PARKIN2 gene to establish a fourth value VPARKIN2;
of the STOX2 gene to establish a fifth value VSTOX2; and
of the amount of mitochondrial DNA to establish a sixth value VADNM;
   and a calculator for:
a. normalizing the values Vₓ to determine six normalized coefficients Cₓ=I Vₓ−VTₓI/VTₓ; and
b. determining the nearest subspace (in the sense of Euclidean distance) of the n-tuple Cₓ to determine the status of the subject.

### BRIEF DESCRIPTION OF THE DRAWINGS

The present disclosure will be better understood on reading the following description, which concerns a non-limiting exemplary embodiment that is shown by the appended drawings.

FIG. 1 shows the distribution of APOE genotypes of patients based on age.

FIG. 2 shows the ROC curve for the detection of patients with the APOE4 genotype via ATM quantification by qPCR.

FIG. 3 shows the ROC curve for detecting patients with the APOE4 genotype via cytoplasmic quantification of the ATM protein.

FIG. 4 shows the distribution of normalized relative ATM gene expression based on patient age in the form of a binary variable (<60 years=1, >60 years=0).

FIG. 5 shows the distribution of ATM protein foci quantification 24 hours post-irradiation based on patient age in the form of a binary variable (<60 years=1, >60 years=0).

FIG. 6 shows the % of cells with normal and abnormal nuclei in Alzheimer's patients.

FIG. 7 shows the analysis of the nuclear morphology of cells from Alzheimer's patients.

FIG. 8 shows a western blot quantifying pATM and Progerin proteins from protein extracts from cells of Alzheimer's patients.

FIG. 9 shows the quantification of ATM, GSNOR and mitochondrial DNA in the cells of Alzheimer's patients.

### DETAILED DESCRIPTION

The aim of the present disclosure is to establish automatically and early on the status of a subject with respect to the probability of occurrence of Alzheimer's disease, for example, to automatically determine the existence of a predisposition to Alzheimer's disease, before the subject develops the disease or the predicted age of the onset of signs of the disease in a subject in respect of Alzheimer's disease.

The first step consists in selecting a set of biomarkers comprising at least three of the following biomarkers, known as genetic risk factors:
1. ATM (Ataxia telangiectasia mutated), a serine/threonine kinase belonging to the family of kinases related to phosphatidylinositol 3-kinase, helping to regulate the response to DNA damage;
2. APOE, (apolipoprotein E);
3. GSNOR (S-nitrosoglutathione reductase), or ADH5, enzyme of the alcohol dehydrogenase (ADH) family;
4. PARKIN2, gene located on human chromosome 6, ubiquitin ligase-type enzyme;
5. STOX2, macaca nemestrina gene; and
6. Mitochondrial DNA.

The selected combination comprises at least three and preferably at least 4 of the above six biomarkers, and may comprise additional biomarkers.

This combination of N biomarkers forms an N-dimensional space that will be used to determine specific subsets characterizing a predisposition status of a subject to a neurodegenerative disease.

This step also consists of defining the assay protocol for each of the N biomarkers selected.

### Preparation of a Repository

The next step consists of testing cohorts of subjects whose predisposition status to a neurodegenerative disease is known, by taking biological samples and then applying the protocol for assaying the N biomarkers selected.

The results of these assays determine, for each subject, a N-tuple of values, associated with a class of predisposition to a known neurodegenerative disease.

The set of N-tuples associated with the same predisposition class defines a domain forming a homogeneous subset of the above-mentioned N-dimensional space.

This repository is not static but is enriched as and when new qualified samples are taken as well as by older samples from subjects whose predisposition status becomes verifiable over time.

### Preparation of a Repository

To determine the predisposition status of a subject, a biological sample is taken.

The sample is preferentially a blood sample, but can also be a skin sample or a sample of cerebrospinal fluid.

The aforementioned N biomarkers are assayed, and an N-tuple defining a point in N-dimensional space is determined. This point is associated with a predisposition status to a neurodegenerative disease of the nearest domain.

### Processing a Sample

By way of non-limiting example, the biomarkers are assayed using a healthy tissue sample, preferably lymphoblasts/lymphocytes or fibroblasts. The first are preferably collected from the blood and the latter from the connective tissue or the skin of an individual. This sample can be collected by blood sampling or biopsy.

The biomarker assay can be carried out by a procedure well known to a person skilled in the art, consisting of:
1—a qPCR analysis of the expression of various biomarkers on coding DNA and comprising:
   a) means for extracting the RNA and synthesizing the coding DNA from the cell sample, that is, from reagents such as a lysis buffer and a RT-PCR kit known to a person skilled in the art; and
   b) means for determining in the coding DNA from the cell sample the expression of certain genes in the basal state (in the state wherein the DNA is found in the cell, without external modification), by using qPCR reagents known to a person skilled in the art and a qPCR apparatus.

The expression information is then analyzed in silico in order to determine the expressions of the genes tested for each individual.
2—An analysis of the nuclear morphology of fibroblasts using specific biomarkers comprising:
   a. Means for visualizing the DNA (DAPI) and the nuclear lamina (anti-lamin A/C antibody); and
   b. Means for confirming the accumulation of biomarkers in the cell (anti-pATM antibodies, anti-progerin antibodies).

### Performing an Assay

A first object of the present disclosure is a method for characterizing the severity of Alzheimer's disease from a sample taken from a patient wherein:
DNA is extracted from a blood or skin sample and the APOE allele is sequenced in order to determine the patient's status;
RNA is extracted followed by synthesis of coding DNA called the “genomic sample”;
the expression of ATM, APOE, GSNOR, PARKIN2 and SOX2 markers and quantification of mitochondrial DNA are determined on this genomic sample; and
the severity of the disease is determined by associating the expression of these markers with the APOE genotype, which makes it possible to separate “young Alzheimer” patients from “old Alzheimer” patients.

A second object of the present disclosure is a method for characterizing the “Alzheimer's patient” status from a skin sample taken from a patient wherein:
the sampled cells are amplified;
the morphology of cell nuclei is determined by staining with DAPI or with a nuclear lamina marker (anti-lamin A/C antibody); and
specific perinuclear accumulation of biomarkers is determined for the ATM protein (presence of perinuclear accumulation) and within the nuclear lamina by immunofluorescence.

Examples: Fibroblasts from 10 patients suffering from the disease were amplified then the genetic material (DNA and RNA) was extracted and a certain number of analyses were carried out by sequencing, qPCR and Immunofluorescence.

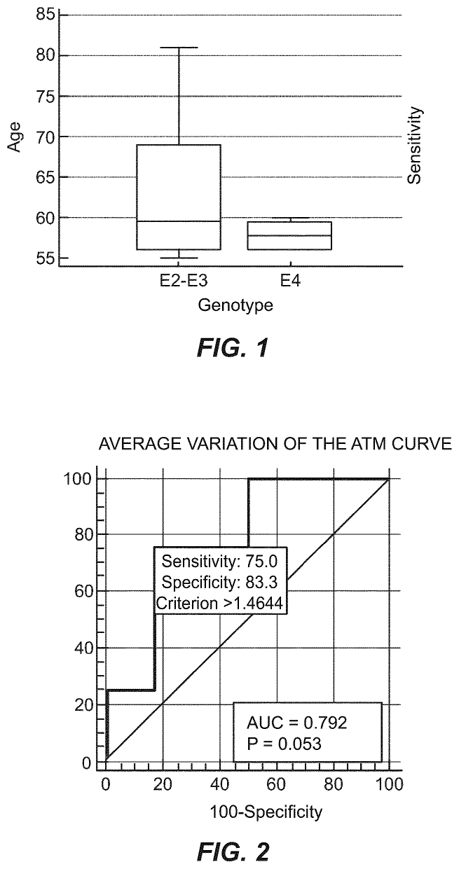

FIGS. 1 to 4 show the ATM quantification curves based on patient genotype. ATM quantification can be used to detect patients having an APOE4 genotype.

FIGS. 1 and 2 show the detection of more severe APOE4 cases via ATM quantification by qPCR on cell lines from 10 Alzheimer's patients.

It was first observed that all patients with an APOE4 genotype (results obtained by sequencing) were under 60 years of age (FIG. 1). Furthermore, the amount of ATM detected by qPCR allows us to identify patients having an APOE4 genotype with a performance of 80% (FIG. 2: ROC curve on the right), and by extension, younger patients (therefore having a more severe phenotype).

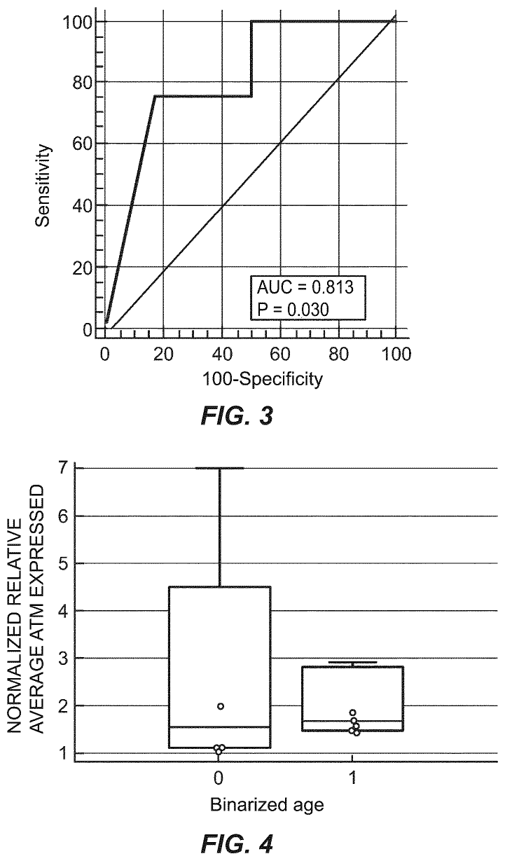

FIG. 3 shows the detection of more severe APOE4 cases by quantifying perinuclear ATM accumulation by immunofluorescence on cell lines from 10 Alzheimer's patients. Furthermore, the number of cells having perinuclear accumulations of pATM detected by immunofluorescence allows us to identify patients having an APOE4 genotype with a performance of 80% (FIG. 2: ROC curve on the right), and by extension, younger patients (therefore having a more severe phenotype).

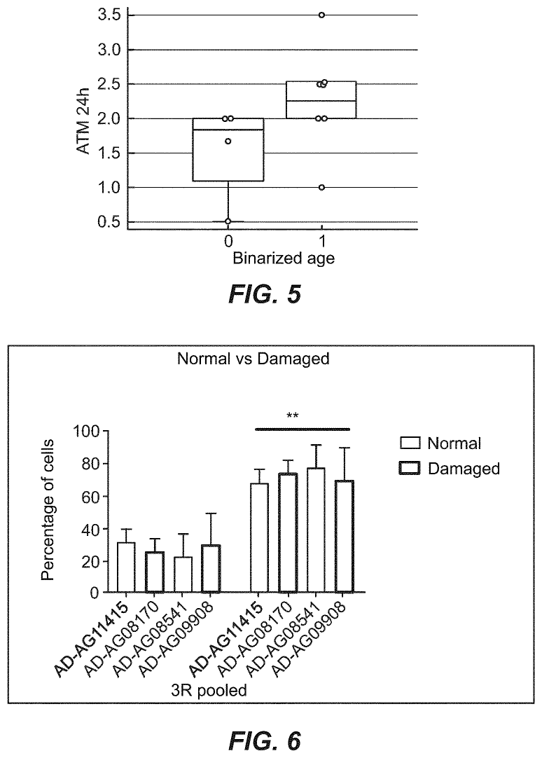

FIGS. 4 and 5 show the distribution of patients based on the age of onset of symptoms (n=10) via ATM quantification by qPCR or the number of pATM foci at 24 hours quantified by immunofluorescence. Alzheimer's patients were divided into 2 classes:
Patients<60 years (1 on FIGS. 4 and 5)
Patients>=60 years (0 on FIGS. 4 and 5)

It can be seen that patients over 60 years of age have the highest ATM expression (FIG. 4). Similarly, the distribution of the number of pATM foci at 24 hours post-irradiation differs based on age (FIG. 5). Younger patients are those with the most foci and therefore defective repair.

FIGS. 6 to 8 show graphs of the increase in morphological abnormalities observed based on the age of the patients.

FIG. 6 shows the number of cells with nuclear abnormalities detected by DAPI staining.

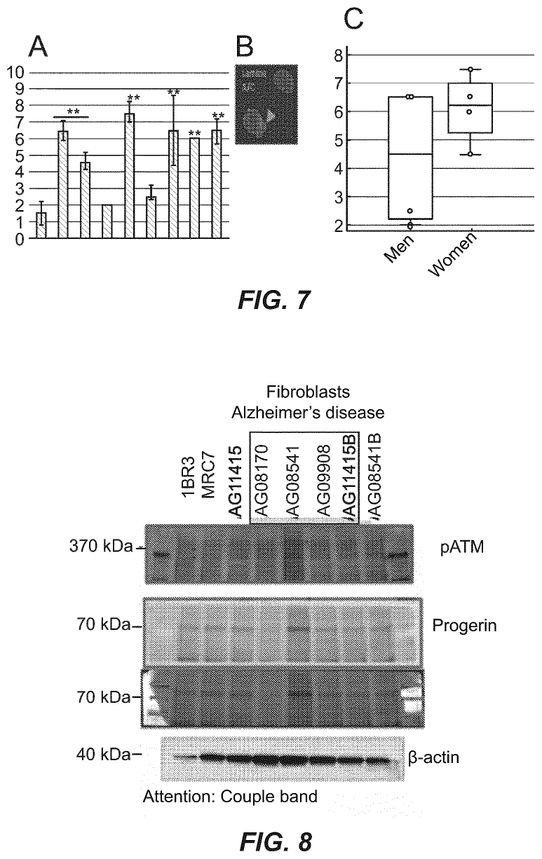

FIG. 7 shows nuclear lamina abnormalities using lamin A/C staining. A. % of cells having nuclear lamina abnormalities (visualized by immunofluorescence with anti-lamin A/C antibody). 100 nuclei counted. **P=0.01 B. Nuclear lamina staining in fibroblast nuclei from an Alzheimer's patient. The arrow indicates the nuclear abnormalities. C. Distribution of % of nuclear lamina abnormalities in fibroblasts from Alzheimer's patients based on gender (p=0.0078).

It is noted that these nuclear lamina abnormalities increased significantly based on the age of patients (Wilcoxon Test p=0.0076) and on the gender of patients (Wilcoxon Test p=0.0078).

FIG. 8 shows the quantification of progerin by western blot in Alzheimer cells. It was observed that all Alzheimer's patients expressed progerin, which is an abnormal version of the Lamin A/C protein contained in the nuclear lamina and which is responsible for the premature aging disease Hutchinson-Gilford Progeria Syndrome (Varela et al., Nature Medicine 2008).

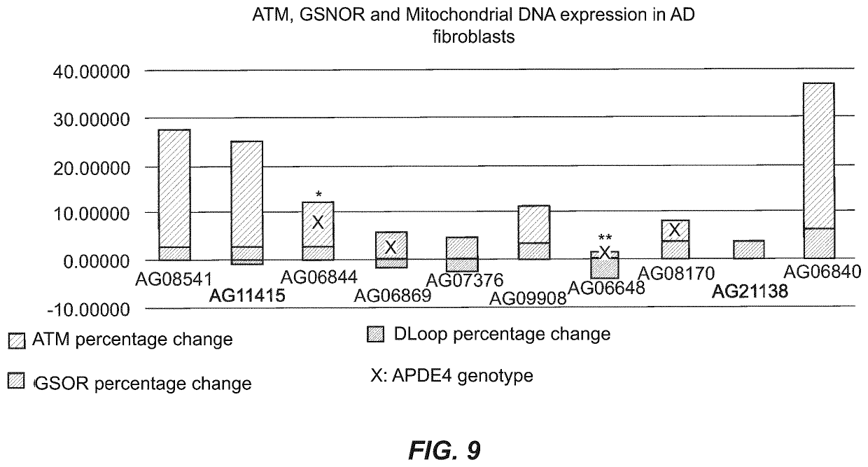

FIG. 9 shows ATM and GSNOR gene expression and mitochondrial DNA quantification in 10 patients with Alzheimer's disease.

It highlights a significant correlation between patients having an APOE4 genotype, and the decrease in the amount of these 3 biomarkers in patients under 60 years of age.

By combining all of these factors, it is possible to use several biomarkers to detect patients under the age of 60 having an APOE4 phenotype and suffering from Alzheimer's disease.

1. A method for automatically classifying the status of a subject in respect of Alzheimer's disease, comprising:
A. an initial step consisting of collecting reference data comprising:
a. a database of reference control values VTₓ for healthy subjects for each of the ATM, APOE, GSNOR, PARKIN2, STOX2, mitochondrial DNA genes; and
b. known status classes, determined from normalized n-tuples CRₓ obtained from biological samples on homogeneous cohorts with respect to one or more classification criteria of patients with a common reference status R to determine a subspace R corresponding to a given status;
B. steps, for a new subject, comprising:
a. using genomic material extracted from a biological sample taken from the new subject, quantifying the following markers:
of the ATM gene to establish an initial value V_{ATM};
of the APOE gene to establish a second value V_{APOE};
of the GSNOR gene to establish a third value V_{GSNOR};
of the PARKIN2 gene to establish a fourth value V_{PARKIN2};
of the STOX2 gene to establish a fifth value V_{STOX2}; and
of mitochondrial DNA to establish a sixth value V_{ADNM};
b. normalizing the values Vₓ to determine six normalized coefficients Cₓ=I Vₓ−VTₓ I/VTₓ; and
c. determining the nearest subspace in the Euclidean distance sense of the n-tuple Cₓ to determine the status of the subject.

2. The method of claim 1, wherein the biological samples are blood samples.

3. The method of claim 1, wherein the biological samples are skin samples.

4. The method of claim 1, wherein the biological samples are brain cell samples or cells from a lumbar puncture.

5. The method of claim 1, further comprising isolating the cells in order to characterize the morphology of the cell nuclei and the level of progerin, to determine the P value of the proportion of cells having a deformed nucleus and to determine the aging status based on the class to which this P value belongs, to be weighted by analyzing the morphology of the nuclei (80% of normal nuclei) by immunofluorescence and antibodies and/or a fluorescent DNA intercalating dye, to confirm the probability of AD, even asymptomatic and/or a higher than normal progerin level.

6. The method of claim 1, further comprising a cell analysis step for verifying the perinuclear presence of phosphoAPOE protein and/or quantifying phosphoAPOE protein.

7. The method of claim 1, further comprising an analysis step by a regression method on an N-tuple of values Vₓ corresponding to the subject to be diagnosed and an N-tuple of control values VTₓ.

8. The method of claim 1, further comprising a step of assaying a pATM-pAPOE biomarker in a skin sample from the subject.

9. The method of claim 8, wherein the assay comprises quantifying the percentage of fibroblasts showing spontaneous and/or post-irradiation perinuclear accumulations of pATM and pAPOE on a significant sample of cells.

10. A kit for qualifying the status of a subject in respect of Alzheimer's disease, according to the method of claim 1, the kit comprising means for quantifying at least some of the following markers:
of the ATM gene and its expression to establish an initial value V_{ATM};
of the APOE gene and its expression to establish a second value V_{APOE};
of the GSNOR gene and its expression to establish a third value V_{GSNOR};
of the PARKIN2 gene and its expression to establish a fourth value V_{PARKIN2};
of the STOX2 gene and its expression to establish a fifth value V_{STOX2}; and
of mitochondrial DNA to establish a sixth value V_{ADNM};
and a calculator for:
a. normalizing the values Vₓ to determine six normalized coefficients Cₓ=I Vₓ−VTₓI/VTₓ I; and
b. determining the nearest subspace (in the sense of Euclidean) distance of the n-tuple Cₓ to determine the status of the subject.
